(19) United States
(12) Patent Application Publication
(10) Pub. No.: US 2025/0009341 A1
(43) Pub. Date: Jan. 9, 2025

(54) SYSTEMS AND METHODS FOR TISSUE ANALYSIS AND VISUALIZATION

(71) Applicant: LUMA Vision Limited, Dublin (IE)

(72) Inventors: Maximilian Ofir, München (DE); Christoph Hennersperger, München (DE)

(21) Appl. No.: 18/763,063

(22) Filed: Jul. 3, 2024

Related U.S. Application Data
(60) Provisional application No. 63/525,262, filed on Jul. 6, 2023.

(51) Int. Cl. A61B 8/08; A61B 8/12

(52) U.S. Cl. CPC A61B 8/481; A61B 8/0891; A61B 8/12; A61B 8/483

(57) ABSTRACT

The invention relates to methods, systems, and devices providing tissue analysis and visualization using contrast enhanced ultrasound.

### CROSS-REFERENCE TO RELATED APPLICATIONS

This application claims priority to, and the benefit of, U.S. Provisional Application No. 63/525,262, filed Jul. 6, 2023, the content of which is incorporated by reference herein in its entirety.

### FIELD OF THE INVENTION

The invention generally relates to ultrasound imaging, and, more particularly, to systems and devices for providing tissue analysis and visualization using contrast enhanced ultrasound.

### BACKGROUND

Ultrasound imaging is a medical imaging technique for imaging organs and soft tissues in a human body. An ultrasound image is produced based on the reflection of high-frequency sound waves off of body structures. The strength (amplitude) of the sound signal in conjunction with the time it takes for the wave to travel through the body provides the information necessary to produce the image.

Ultrasound imaging can help a physician evaluate, diagnose and treat various medical conditions. When making a diagnosis based on an ultrasound examination, physicians must rely on adequate image quality, acquisition of proper views, and sufficient quantification of all relevant structures and flows.

For example, catheter-based endovascular ultrasound imaging technology employed within the vasculature (e.g., intravascular ultrasound (IVUS) or intracardiac echocardiography (ICE)) is commonly performed with two-dimensional (2D)-ultrasound imaging. In IVUS/ICE imaging systems, an ultrasonic transducer assembly is attached to a distal end of a catheter. The catheter is carefully maneuvered through a patient's body to an area of interest, such as within a coronary artery (for the case of IVUS), or within the right atrium (for the case of ICE). The transducer assembly transmits ultrasound waves and receives echoes from those waves. The received echoes are then converted to electrical signals and transmitted to processing equipment, in which a resulting ultrasound image of the area of interest may be displayed.

In typical ultrasound systems configured to visualize inner body regions, dynamic forces are often employed, resulting in a dynamic movement of the body regions over time. These dynamic forces and movements make it difficult to stabilize internal imaging devices and to generate consistent and accurate images if imaging of the structure cannot be enabled in real-time (e.g., >20 Hz). As a result, the captured images often lack the necessary quality required to prescribe appropriate treatment or therapy. Because of the dynamic forces and movements in play, internal real-time imaging is limited to small two-dimensional areas or limited three-dimensional volumetric regions respectively.

For certain treatments, such as catheter ablation, accurately capturing a visual representation of the anatomy of interest is paramount for a successful procedure. Catheter ablation is a treatment in which energy is applied to cardiac tissue to create scars or lesions for preventing or interrupting the transmission of abnormal electrical signals. Catheter ablation forms an essential part of the management of cardiac arrhythmias, including supraventricular tachycardia (SVT), atrial flutter (AFL), atrial fibrillation (AF) and ventricular tachycardia (VT). The relatively low efficacy of AF treatment is likely due to limitations in mapping, incomplete understanding of the driving mechanisms of arrhythmia, and, most importantly, the inability to create transmural and durable lesions.

Successful catheter ablation requires not only precise localization of the arrhythmogenic substrate, but complete and permanent elimination of that substrate without producing collateral injury. The ablation effect depends on a number of factors, including applied electrical power, quality of the electrical contact, local tissue properties, presence of blood flow close to the tissue surface, and the effect of irrigation. Because of the variability of these parameters, it may be difficult to obtain consistent results and understand ablation effects in tissue using current systems and methods for ablation. As a result, current ablation systems may be limited because of the difficulties and challenges in tissue analysis and imaging before and after an ablation procedure.

### SUMMARY

The present invention recognizes the limitations of current tissue analysis and visualization using ultrasound technology, namely the inability to image and analyze the transmurality and continuity of ablation paths.

In particular, the invention provides systems and methods for real-time, contrast-enhanced ultrasound imaging of an anatomical region of interest that uses data related to changes in the microvasculature to provide improved tissue imaging. More specifically, the present invention utilizes three-dimensional (3D) and/or four-dimensional (4D) ultrasound images of the anatomy of interest, as opposed to 2D ultrasound imaging, to facilitate detection and visualization of the full extent of performed ablations in the anatomy by exploiting characteristic changes in the microvasculature associated therewith, specifically utilizing changes in ultrasound data related to tissue perfusion to indicate/detect and visualize lesions.

For example, systems and methods of the present invention may generally receive ultrasound image data (i.e., 3D and/or 4D images) from an imaging device, such as an US imaging machine. The ultrasound image data is generally associated with an anatomical region of interest including a targeted tissue site, such as a specific site to undergo ablation therapy (i.e., cardiac tissue or the like). The ultrasound image data is further associated with perfusion of a contrast agent with the targeted tissue site before, during, and/or after an ablation procedure is performed thereon. Upon analysis of perfusion of the contrast agent relative to vasculature associated with the targeted tissue site, multiple images from the ultrasound image data are dynamically reconstructed to provide a 3D visualization of the anatomical region of interest and targeted tissue site, including, among other things, visualization of lesion formations in the targeted tissue site.

The analysis of perfusion of the contrast agent includes identifying perfusion characteristics in microvasculature of the targeted tissue site, wherein a given lesion formation is identified based on such perfusion characteristics. For example, unobstructed propagation and accumulation of a contrast agent into a given location of microvasculature can be generally indicative of unaffected and otherwise healthy microvasculature, while a lack of propagation and accumulation of a contrast agent into a given location is indicative of damaged microvasculature. Accordingly, the systems and methods of the present invention are able to effectively identify and characterize a lesion formation based, at least in part, on correlation of perfusion characteristics with physical characteristics of a given location of the microvasculature. In turn, visualization of a given lesion formation may include an extent of the lesion formation, transmurality of the lesion formation, and continuity of an ablation path associated with the lesion formation.

Accordingly, the invention provides improved lesion analysis, particularly for catheter-induced ablation procedures. Current imaging techniques lack the ability to measure the extent, transmurality and continuity of ablation paths induced by ablation catheters during ablation procedures. Each of these attributes correlates with long-term isolation success in cardiac arrhythmia treatments. The present invention recognizes that tissue perfusion can be used in identifying and/or detected ablated regions. As such, the present invention is able to utilize tissue perfusion, namely real-time, contrast-enhanced ultrasound imaging to identify changes in the microvasculature of a tissue of interest, and provide visual evidence of the extent of an identified lesions, prior to, during, and after surgery, thereby allowing improved lesion path planning and execution, and improved patient outcomes.

Aspects of the present invention include systems for providing tissue analysis and visualization that include a console operably associated with an imaging device. In particular, the imaging device includes a a console comprising a hardware processor coupled to non-transitory, computer-readable memory containing instructions executable by the processor to cause the console to receive three-dimensional (3D) ultrasound image data from an imaging device, and dynamically reconstruct multiple images from the 3D image data. The 3D image data is associated with an anatomical region of interest including a targeted tissue site and perfusion of a contrast agent therewith before, during, and/or after an ablation procedure being performed on the tissue. The dynamically reconstructed images provide a 3D visualization of an anatomical region of interest and targeted tissue site, based, at least in part, on analysis of perfusion of the contrast agent relative to vasculature associated at least with the targeted tissue site.

In some embodiments of the system, the 3D ultrasound image data is real-time 3D ultrasound data. The 3D visualization comprises, in some embodiments, visualization of lesion formations in the targeted tissue site.

In particular embodiments, the tissue includes microvasculature associated with the targeted tissue site. For example, the analysis may include identifying perfusion characteristics in the microvasculature. Further, a lesion formation is, in some embodiments, identified based on the perfusion characteristics. In some embodiment of the systems, the console is configured to correlate perfusion characteristics within a given location of the microvasculature with physical characteristics of the microvasculature at said given location. In specific embodiments, the perfusion characteristics include a plurality of gradations of propagation and accumulation of contrast agent into a given location of microvasculature.

For example, in some embodiments, unobstructed propagation and accumulation of contrast agent into a given location of microvasculature is indicative of unaffected and otherwise healthy microvasculature, and lack of propagation and accumulation of contrast agent into a given location is indicative of damaged microvasculature. Specifically, the damaged microvasculature, in particular embodiments, is a result of ablation and the lack of propagation and accumulation of contrast agent into the given location is indicative of a portion of a lesion formation. Accordingly, in some embodiments of the systems, the console is configured to characterize a lesion formation based, at least in part, on correlation of the perfusion characteristics with physical characteristics of a given location of the microvasculature. These physical characteristics may be one or more of flow, microflow, and stiffness, in some embodiments. In specific embodiments of the systems of the invention, characterization includes, providing a visual indication of at least one of an extent of the lesion formation, transmurality of the lesion formation, and continuity of an ablation path associated with the lesion formation.

Further, in some embodiments of the systems, the console is configured to segment a given lesion formation into at least three different regions comprising a core region, a border region immediately adjacent to and surrounding the core region, and a periphery region immediately adjacent to and surrounding the border region. For example, in particular embodiments, the core region of a lesion formation is associated with a complete, or near complete, lack of propagation and accumulation of contrast agent into a given location of the microvasculature and appears normal within a 3D ultrasound image. A border region of a lesion formation is associated with some propagation and accumulation of contrast agent into a given location of the microvasculature and presents a stronger backscatter signal within a 3D ultrasound image as compared to a backscatter signal associated with the core region. Further, a periphery region of a lesion formation is associated with substantially unobstructed propagation and lack of accumulation of contrast agent into a given location of microvasculature and presents a weaker backscatter signal within a 3D ultrasound image as compared to backscatter signals associated with the border region a short time after injection, in some embodiments.

In some examples of systems of the invention, the console performs segmentation of a given lesion formation based, at least in part, on a segmentation algorithm. For example, in specific embodiments, the segmentation algorithm comprises at least one of automatic thresholding, connected component analysis, and neural network-based segmentation.

In some embodiments, the contrast agent may be injected into vasculature before and/or after performing one or more ablation procedures. Specifically, in some embodiments, the systems of the invention further comprise a catheter-based ultrasound imaging device operably coupled to the console and configured to transmit ultrasound pulses to, and receive echoes of the ultrasound pulses from, intravascular and/or intracardiac tissue. For example, the ultrasound imaging device may comprise a four-dimensional (4D) catheter-based ultrasound imaging device, in some embodiments. Accordingly, the console may be configured to receive at least full circumferential, 3D image data from the ultrasound imaging device in real, or near-real time, and the console is configured to reconstruct multiple images in real, or near-real time, based, at least in part, on user input and/or predefined protocols. The console may be configured to provide a 3D visualization of the anatomical region of interest and targeted tissue site during an ablation procedure being performed on the targeted tissue site, in specific embodiments.

In specific embodiments of systems of the invention, the anatomical region of interest and targeted tissue site are associated with myocardial tissue.

In further aspects, the invention discloses methods for providing lesion analysis and visualization. The methods include, in some embodiments, providing a console configured to be operably coupled to an imaging device and to communicate and exchange data therewith, receiving, via the console, three-dimensional (3D) ultrasound image data from the imaging device, and dynamically reconstructing, via the console, multiple images from the 3D image data to provide a 3D visualization of the anatomical region of interest and targeted tissue site. The 3D image data is associated with an anatomical region of interest including a targeted tissue site and perfusion of a contrast agent therewith before, during, and/or after an ablation procedure being performed thereon. The 3D visualization comprises visualization of any detected lesion formations in the targeted tissue site based, at least in part, on analysis of perfusion of a contrast agent relative to vasculature associated at least with the targeted tissue site.

In some embodiments of the methods, the 3D ultrasound image data is real-time 3D ultrasound data. The 3D visualization comprises, in some embodiments, visualization of lesion formations in the targeted tissue site.

In particular embodiments of the methods, the tissue includes microvasculature associated with the targeted tissue site. For example, the analysis may include identifying perfusion characteristics in the microvasculature. Further, a lesion formation is, in some embodiments, identified based on the perfusion characteristics.

In some embodiment of the methods, the console is configured to correlate perfusion characteristics within a given location of the microvasculature with physical characteristics of the microvasculature at said given location. In specific embodiments, the perfusion characteristics include a plurality of gradations of propagation and accumulation of contrast agent into a given location of microvasculature.

For example, in some embodiments of the methods, unobstructed propagation and accumulation of contrast agent into a given location of microvasculature is indicative of unaffected and otherwise healthy microvasculature, and lack of propagation and accumulation of contrast agent into a given location is indicative of damaged microvasculature. Specifically, the damaged microvasculature, in particular embodiments, is a result of ablation and the lack of propagation and accumulation of contrast agent into the given location is indicative of a portion of a lesion formation. Accordingly, in some embodiments of the methods, the console is configured to characterize a lesion formation based, at least in part, on correlation of the perfusion characteristics with physical characteristics of a given location of the microvasculature.

These physical characteristics may be one or more of flow, microflow, and stiffness, in some embodiments of the method. In specific embodiments of the methods of the invention, characterization includes, providing a visual indication of at least one of an extent of the lesion formation, transmurality of the lesion formation, and continuity of an ablation path associated with the lesion formation.

Further, in some embodiments of the methods, the console is configured to segment a given lesion formation into at least three different regions comprising a core region, a border region immediately adjacent to and surrounding the core region, and a periphery region immediately adjacent to and surrounding the border region. For example, in particular embodiments, the core region of a lesion formation is associated with a complete, or near complete, lack of propagation and accumulation of contrast agent into a given location of the microvasculature and appears normal within a 3D ultrasound image. A border region of a lesion formation is associated with some propagation and accumulation of contrast agent into a given location of the microvasculature and presents a stronger backscatter signal within a 3D ultrasound image as compared to a backscatter signal associated with the core region. Further, a periphery region of a lesion formation is associated with substantially unobstructed propagation and lack of accumulation of contrast agent into a given location of microvasculature and presents a weaker backscatter signal within a 3D ultrasound image as compared to backscatter signals associated with the border region a short time after injection, in some embodiments.

In some examples of methods of the invention, the console performs segmentation of a given lesion formation based, at least in part, on a segmentation algorithm. Specifically, in some embodiments, the segmentation algorithm comprises at least one of automatic thresholding, connected component analysis, and neural network-based segmentation.

In specific representations of the methods, the contrast agent may be injected into vasculature before and/or after performing one or more ablation procedures. For example, in some embodiments, the systems of the invention further comprise a catheter-based ultrasound imaging device operably coupled to the console and configured to transmit ultrasound pulses to, and receive echoes of the ultrasound pulses from, intravascular and/or intracardiac tissue. Specifically, in some examples, the ultrasound imaging device may comprise a four-dimensional (4D) catheter-based ultrasound imaging device, in some embodiments. Accordingly, the console may be configured to receive at least full circumferential, 3D image data from the ultrasound imaging device in real, or near-real time, and the console is configured to reconstruct multiple images in real, or near-real time, based, at least in part, on user input and/or predefined protocols. The console may be configured to provide a 3D visualization of the anatomical region of interest and targeted tissue site during an ablation procedure being performed on the targeted tissue site, in specific embodiments.

In specific embodiments of methods of the invention, the anatomical region of interest and targeted tissue site are associated with myocardial tissue.

In another aspect, the invention provides methods for contrast-enhanced imaging comprising the steps of injecting the contrast agent, receiving 3D image data, dynamically reconstructing the image data, providing a 3D visualization of an anatomical region of interest, and monitoring the perfusion of the contrast agent. In some embodiments, the contrast agent is injected directly into the heart. In other embodiments, the contrast agent is injected indirectly through a vein or an artery. Injection of the contrast agent, in some embodiments, may occur one or more times, for example as it relates to imaging an anatomical region of interest and/or to suit the needs of the procedure performed. Perfusion of the contrast agent is monitored via ultrasound imaging. In some embodiments, monitoring may include monitoring the flush of the contrast agent, the perfusion of the contrast agent, and/or the dispersion of the contrast agent. Further, in some embodiments, monitoring via ultrasound imaging may performed at any time, for example at injection of the contrast agent, before and/or during a procedure, and post-follow-up after a procedure. In some embodiments, monitoring may include displaying changes in perfusion of the contrast agent. In some embodiments, reconstructing may include reconstructing multiple images from the 3D data to provide a 3D visualization of the anatomical region of interest and targeted tissue site. Accordingly, in some embodiments, reconstruction may include algorithmically relating changes in the contrast agent perfusion to tissue properties.

In another aspect, the invention provides a method for contrast-enhanced imaging comprising the steps of injecting the contrast agent, receiving 3D image data, segmenting an anatomical region of interest, displaying a visualization of changes in perfusion of the contrast agent, and relating the changes in perfusion of the contrast agent to tissue properties.

### BRIEF DESCRIPTION OF THE DRAWINGS

FIGS. 1A and 1B are diagrammatic illustrations of an exemplary ultrasound system for providing visualization and characterization of vasculature within a patient with which devices of the invention may be used.

FIG. 2 is a perspective view of an imaging catheter with which devices of the invention may be coupled.

FIG. 3 illustrates an embodiment of a system the invention in which the console is in active communication with a computing system comprising an algorithm for assessing, visualizing, and/or evaluating a targeted tissue site.

FIG. 4 is a block diagram of a method for providing lesion analysis and visualization according to one embodiment of the invention.

FIG. 5 is block diagram of a method for contrast-enhanced imaging according to one embodiment of the invention.

FIG. 6 is a block diagram of a method for the contrast-enhanced imaging according to one embodiment of the invention.

### DETAILED DESCRIPTION

The present invention recognizes the limitations of current tissue analysis and visualization using ultrasound technology, namely the inability to image and analyze the transmurality and continuity of ablation paths. In particular, the invention provides novel systems and methods for real-time, contrast-enhanced ultrasound imaging of an anatomical region of interest. Specifically, characteristic changes in ultrasound data related to changes in the vasculature or microvasculature are exploited to provide improved tissue imaging. In particular embodiments, the invention provides novel systems and methods for lesion imaging, however, it is to be understood that systems and methods of the invention provide for improved tissue imaging in general.

For example, the novel systems and methods of the invention provide for measuring the extent, transmurality and continuity of ablation paths induced by ablation catheters during ablation procedures, which is currently not possible with contemporary systems. Each of these attributes correlates with long-term isolation success in cardiac arrhythmia treatments. Thus, the invention provides real-time visual information regarding the extent of the lesions thereby allowing better lesion path planning and execution, and improved patient outcomes.

Systems and methods of the invention use an ultrasound contrast agent to visualize tissue qualities through a change in perfusion in the tissue evaluated. In specific examples, the invention discloses visualizing an area of ablation through a change in perfusion to tissue such as myocardial tissue before, during, and/or after an ablation procedure.

Aspects of the invention disclose a system for providing tissue analysis and visualization. The system may include a console with a hardware processor coupled to non-transitory, computer-readable memory containing instructions executable by the processor. The instructions may to cause the console to receive three-dimensional (3D) ultrasound image data from an imaging device, and dynamically reconstruct multiple images from the 3D image data to provide a 3D visualization of the anatomical region of interest and targeted tissue site. The 3D image data may be associated with an anatomical region of interest including a targeted tissue site. The targeted tissue site may have an associated level of perfusion when applied, for example, before, during, and/or after an ablation procedure being performed upon the tissue. The reconstructed image(s) may be based, at least in part, on analysis of the level of perfusion of the contrast agent relative to vasculature associated at least with the targeted tissue site.

### Ultrasound Imaging System

By way of overview, and as is generally understood, ultrasound imaging (sonography) uses high-frequency sound waves to view inside the body. Because ultrasound images are captured in real-time, these images can also show movement of the body's internal organs as well as fluid flow (e.g., blood flowing through blood vessels). In an ultrasound exam, the imaging device, (i.e. the transducer, probe, or transducer probe) is placed directly on the skin or inside a body opening (e.g. endovascular ultrasound, intravascular ultrasound, intracardiac echocardiography). The final quality of the image obtained through ultrasound scanning is limited to the technical specifications of the equipment, the propagation of ultrasonic waves through the tissue analyzed, and the method used to reconstruct the images.

Systems of the invention may be operably connected with an ultrasound system with certain hardware and software for providing image reconstruction and imaging assembly control, for example as described in International PCT Application No. PCT/IB2019/000963 (Published as WO 2020/044117) to Hennersperger et al., U.S. Application Publication No. US 2022-0287679A1 to Hennersperger et al., and U.S. Pat. No. 11,382,599 to Hennersperger et al., the contents of each which are incorporated by reference herein. The data may be processed using imaging protocols to extract anatomical and functional information, and tissue characteristics as disclosed in International PCT Application No. PCT/IB2019/000963 (Published as WO 2020/044117) to Hennersperger et al., U.S. Application Publication No. US 2022-0287679A1 to Hennersperger et al., and U.S. Pat. No. 11,382,599 to Hennersperger et al., the contents of each which are incorporated by reference herein.

Systems of the invention are configured to receive three-dimensional (3D) ultrasound image data from an imaging device. In some embodiments, the invention provides for three-dimensional visualization and tissue characterization for use in minimally invasive procedures in the vasculature. Accordingly, ultrafast ultrasound imaging techniques, such as planewave or diverging wave imaging, may be required to enable imaging within the constraints of the application, particularly for intravascular and/or intracardiac tissue assessment and analysis. These constraints could be posed due to the high temporal update rate as required for effects observed in visualization and tissue characterization, where plane and diverging wave methods enable high imaging rates, commonly also referred to ultrafast imaging approaches. Systems and methods of the invention allow for the direct utilization of all native ultrafast imaging techniques.

For example, for intracardiac imaging, planewave imaging may refer to an ultrasound imaging modality where, through a flat transmit of all transducer elements (at different angles) from the angular imaging aperture, a plane wave front may traverse the tissue and may be partially scattered back to the transducer. From the received radio frequency (RF) (i.e. channel) data the overall image may be reconstructed at once in parallel by dynamically beamforming the received RF data for each target position.

Ultrafast ultrasound methods offer imaging at thousands of frames per second limited only by the physical propagation speed of sound waves in tissue, and enable ultrasensitive blood-flow tracking, shear-wave imaging, super-resolution imaging, and other applications. For example, achieving optimal spatial resolution while enabling artifact-free imaging of dynamic cardiac structures requires a careful balance between spatial sampling and volumetric update rate which can only be achieved using ultrafast imaging techniques. Thus, the 3D ultrasound image data received by systems of the invention may be real-time 3D ultrasound data. For example, the data may be full circumferential, three-dimensional (3D) image data. Specifically, the 3D visualization may be visualization of lesion formations in the targeted tissue site.

Systems of the invention may include a catheter-based ultrasound imaging device operably coupled to the console and configured to transmit ultrasound pulses to, and receive echoes of the ultrasound pulses from, intravascular and/or intracardiac tissue.

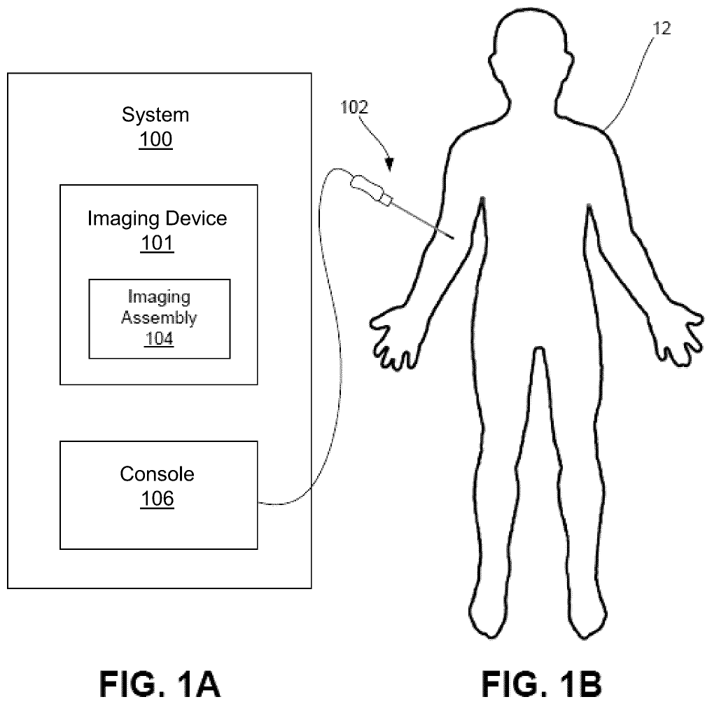

FIG. 1A and FIG. 1B are diagrammatic illustrations of an exemplary system 100 for providing visualization and characterization of tissue and/or vasculature/microvasculature within a patient 12. The system 100 may include an imaging device 101 equipped with an imaging assembly 104 and a console 106 to which the imaging device 101 is to be connected. The imaging device may be an imaging catheter 102. Accordingly, systems and methods for tissue analysis/assessment disclosed herein may use a 4D Intracardiac echocardiogram (ICE) system that captures the anatomy of interest. ICE is a catheter-based form of echocardiography that gathers images from within the heart, rather than by gathering images of the heart by sending sound waves through the chest wall. In specific embodiments of the systems, the ultrasound imaging device comprises a four-dimensional (4D) catheter-based ultrasound imaging device.

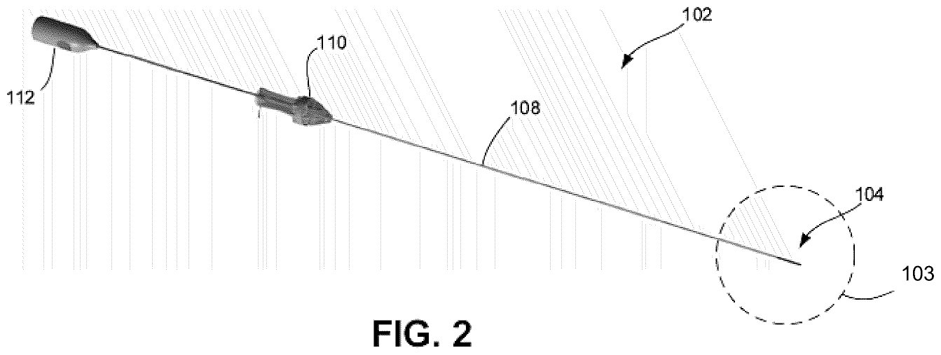

FIG. 2 is a perspective view of an imaging catheter 102 with which systems of the invention may be coupled. The catheter 102 may include a catheter body 108, including proximal and distal portions. The imaging assembly 104 may be provided at the distal portion, for example, generally defining a distal end of an imaging catheter. A handle 110 may be operably associated with the catheter body 108 and allow for an operator (i.e., surgeon or other medical professional) to manipulate and advance the imaging assembly 104 and the catheter body 108 to a desired target site within the patient's vasculature. The handle 110 may include user-operable inputs for controlling various features and functions of the imaging assembly 104. An interface member 112 may be provided at a distal portion of the catheter body 108. The interface member 112 generally provides a connection between the imaging catheter 102, including the imaging assembly 104 and handle 110, and the console 106 for transmission of signals therebetween. The connection may include at least one of a hardwired and wireless connection, for example.

In certain embodiments of the systems, the console is configured to receive at least full circumferential, 3D image data from the ultrasound imaging device in real, or near-real time. Thus, the console is configured to reconstruct multiple images in real, or near-real time, based, at least in part, on user input and/or predefined protocols. As described in more detail herein, in some examples, the console is configured to provide a 3D visualization of the anatomical region of interest and targeted tissue site during an ablation procedure being performed on the targeted tissue site.

The console may be operably coupled to the imaging device and may generally control operation of the transducer probe i.e., transmission of sound waves from the probe. The console may generally include one or more processors (e.g., a central processing unit (CPU), a graphics processing unit (GPU), or both) and storage, such as main memory, static memory, or a combination of both, which communicate with each other via a bus or the like. The memory according to embodiments of the invention can include a machine-readable medium on which is stored one or more sets of instructions (e.g., software) embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

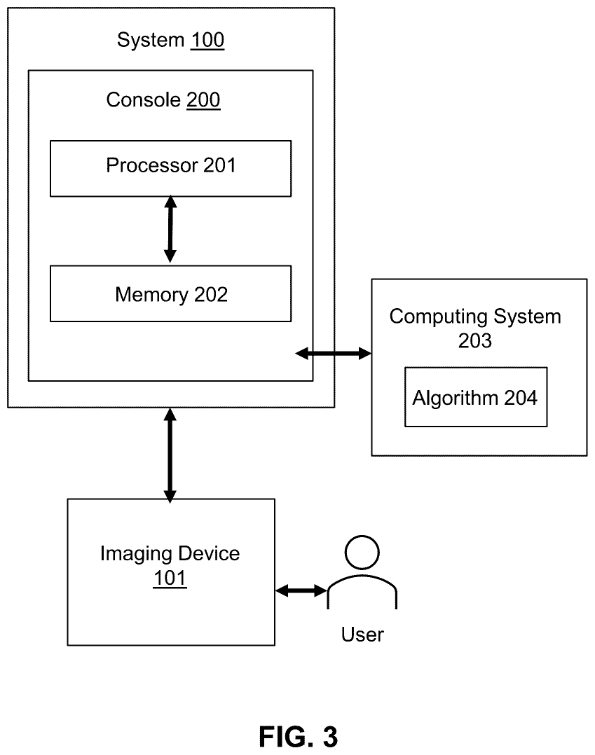

FIG. 3 illustrates one embodiment of systems 100 of the invention. For example, in an exemplary embodiment, the system 100 may include a console 200 in active communication with a computing system 203 configured to communicate across a network. The computing system 203 or computing device may include one or more processors and memory, as well as an input/output mechanism (i.e., a keyboard, knobs, scroll wheels, or the like) with which an operator can interact so as to operate the machine, including making adjustments to the transmission characteristics of the probe, saving images, and performing other tasks described herein, including selection of specific regions of interest for subsequent reconstruction into 2D and/or 3D images.

During operation, the CPU and/or GPU may control the transmission and receipt of electrical currents, subsequently resulting in the emission and receipt of sound waves from the probe. The CPU and/or GPU may also analyze electrical pulses that the probe makes in response to reflected waves coming back and then may converts this data into images (i.e., ultrasound images) that can then be viewed on a display, which may be an integrated monitor. Such images may also be stored in memory and/or printed via a printer. The console may further provide control over an imaging assembly, including control over the emission of ultrasound pulses therefrom (intensity, frequency, duration, etc.) as well as control over the movement of the ultrasound transducer unit.

The computing system 203 may include a computer program comprising an algorithm 204 for assessing and analyzing a targeted tissue site. For example, the algorithm 204 may be part of a computer program executable by the computing system 203 and in communication with the console of the system 100. The system may be in communication with the imaging device 101 to receive 3D ultrasound image data from the imaging device 101.

Imaging protocols and algorithms may be used to reconstruct properties of the targeted tissue site. As discussed in more detail herein, the system may include one or more algorithms for dynamically reconstructing multiple images from the 3D image data to provide a 3D visualization of the anatomical region of interest and targeted tissue site. For example, using defined algorithms, the system may be configured to generate the 3D visualizations based, at least in part, on analysis of perfusion of the contrast agent relative to vasculature and/or microvasculature associated at least with the targeted tissue site.

The evaluation of the vasculature/microvasculature and tissue characteristics using systems of the invention may include both the anatomical depiction of the vasculature/microvasculature, as well as information about the path and depth of lesions during cardiac interventions such as ablation. Specifically, data related to perfusion characteristics may be used to analyzed for visualization of an anatomical region of interest. Tissue reconstruction may be accomplished by also capturing data related to the extraction of perfusion, stiffness, strain, anisotropy, coherence, specific statistical distributions in tissue (Rayleigh, Nakagami), spectral parameters of tissue (frequency power spectrum) and other parameters. This data may be captured using a rotating three-dimensional multi-element ultrasound transducer array. Data may be captured either at a single point in time, or at different stages, for example before, during, or after, an ablation procedure.

The tissue state mapping or functional imaging may be performed by integration of the tissue data with the appropriate imaging protocols and reconstruction algorithms, as described above. These individual protocols and algorithms may be integrated to evaluate and extract information from the data on, for example, stiffness, micro-vasculature, elasticity, perfusion, flow, shear wave speed, and other information that indicates the tissue state.

### Tissue Assessment Using Perfusion Characteristics

As noted above, systems of the invention dynamically reconstruct multiple images from 3D image data to provide a 3D visualization of an anatomical region of interest and targeted tissue site. The 3D visualization provides a means for assessing and analyzing an anatomical region of interest and a targeted tissue site. The assessment is based, at least in part, on analysis of perfusion of a contrast agent relative to vasculature or microvasculature associated at least with the targeted tissue site. Specifically, the invention provides for automated lesion segmentation using 3D/4D data acquisitions of an anatomy of interest thus facilitating detection and visualization of the full extent of lesions in the anatomy.

Prior art approaches to lesion imaging attempt to characterize changes in ultrasound data that are related to tissue property changes, such as stiffness and echogenicity, with little success. The invention discloses a novel approach to imaging scarring and/or necrosis of tissue by exploiting characteristic changes in the microvasculature. Specifically, the systems and methods of the invention use tissue perfusion imaging to indicate and/or detect regions of scar and/or necrosis.

As disclosed, the 3D ultrasound image data may be real-time 3D ultrasound data, and the 3D visualization may include visualization of lesion formations in the targeted tissue site. Thus, the tissue assessment systems and methods of the invention may use a 4D ICE system that captures the anatomy of interest using ultrasound imaging. Specifically an ultrasound contrast agent is used to visualize a targeted tissue site through a change in perfusion to the tissue.

Ultrasound contrast agent is comprised of small echogenic bubbles in the size of red blood cells, as noted in Greis, 2011, Quantitative evaluation of microvascular blood flow by contrast-enhanced ultrasound (CEUS); Clinical hemorheology and microcirculation 49.1-4:137-149, incorporated in its entirety herein. Generally, ultrasound contrast agents consist of tiny gas-filled microbubbles the size of red blood cells. As is known to persons skilled in the art, due to their size distribution, they are purely intravascular tracers which do not extravasate into the interstitial fluid, and thus may be used for imaging blood distribution and flow. Thus, the contrast agent can effectively propagate into both larger vessels and the microvasculature. More generally, the contrast agent can propagate into any vessel where red blood cells can be present.

Ultrasound contrast agents alter the reflection pattern, for example by dramatically increasing backscatter signal, and resonating in a linear manner when acoustic pressure is applied. With increasing acoustic pressure, nonlinear vibrational patterns appear. Specifically, the microbubbles oscillate when being compressed by the effect of positive pressure created by the ultrasound waves and they expand in the negative pressure phase. The compression of the gas is greater than expansion which creates a non-linear response (echo). This greatly affects ultrasound backscatter and increases vascular contrast.

The current invention exploits these properties to provide novel systems and methods for using tissue perfusion, e.g. blood flow in an anatomical region of interest, to detect regions of scar or necrosis in the tissue. Specifically, an ultrasound contrast agent is used to visualize tissue properties through a change in perfusion to tissue. Differences in perfusion within the microvasculature are exploited for novel tissue analysis and visualization.

As disclosed herein, the invention provides a system for receiving three-dimensional (3D) ultrasound image data from an imaging device. The console may be configured to receive at least full circumferential, 3D image data from the ultrasound imaging device in real, or near-real time. Further, the console may be configured to reconstruct multiple images in real, or near-real time, based, at least in part, on user input and/or predefined protocols.

The system provides for dynamically reconstructing multiple images from the 3D image data to provide a 3D visualization of the anatomical region of interest and targeted tissue site. The 3D image data may be associated with an anatomical region of interest including a targeted tissue site and perfusion of a contrast agent within the targeted tissue site. Thus, in some embodiments, the 3D visualization includes visualization of lesion formations in the targeted tissue. For example, the tissue may be microvasculature associated with the targeted tissue site.

The console may be configured to provide a 3D visualization of the anatomical region of interest and targeted tissue site during an ablation procedure being performed on the targeted tissue site. It is important to note that, while specific examples of systems of the invention are directed toward analysis and assessment of ablated regions of the myocardium, the targeted tissue may be any tissue wherein perfusion characteristics of a contrast agent may be evaluated. As described in detail herein, the distribution and/or lack of microvasculature perfusion may be assessed as a marker for tissue in general, including but not limited to, lesion imaging.

Specifically, following propagation of the contrast agent microbubbles in the tissue, an area of scarring and/or necrosis, i.e. a lesion, referred to as the core, will not contain significant amounts of contrast agent (microbubbles) and appear normal in the ultrasound image. A second area, that neighbors the core, referred to as the border region, may be characterized by some thermal damage, and ruptures in the microvasculature where the contrast agent is accumulated in the tissue, thus creating a stronger backscattered signal on the ultrasound image. Systems of the invention capture the changes in perfusion of the contrast agent and algorithmically relate these changes to the tissue properties. Accordingly, in some embodiments, the analysis includes identifying perfusion characteristics in the microvasculature.

In specific embodiments, the system algorithmically segments the lesion into different segments, for example, three or more segments. The segments may be, for example, the core, the border region, and the periphery. The core may be delineated from the brighter-appearing border region, and the unaffected healthy region in the periphery of the lesion. Reconstructed images of these three regions may be identified using systems of the invention, for example using a 3D/4D ultrasound system in combination with the contrast agent. Thus, although the contrast agent in the intact vasculature disappears quickly, the particles that exit through ruptured vasculature remain long enough for imaging.

The segmentation may be performed by a vision algorithm such as thresholding, connected component analysis, or a neural network based segmentation.

The lesions may be visualized in real time. For example, the contrast agent may be injected when the catheter is positioned to view the anatomy of interest. Thus, the anatomy of interest may be viewed while the contrast agent is injected. Accordingly, it is possible to create a 3D anatomical map that shows the tissue before and after an ablation procedure. This allows for a comparative view. In this application, the ultrasound data may be gated using electrocardiogram (ECG) signals and 3D maps computed before and after the ablation of the anatomy of interest

For injection of contrast agent into the blood stream, an injection of the contrast agent directly within the heart or coronary arteries may be made. An injection directly into the coronary arteries enables a quick and efficient distribution of the contrast agent specifically into the myocardium. Alternatively, the contrast agent may be introduced within the left atrium. This enables propagation of the contrast agent from the left atrium (LA) (enabling propagation from the LA to left ventricle (LV) to Aorta and from the aorta into the myocardium), directly in the Aorta, or other anatomical site which promote a transition of microbubbles into the myocardium, as described in Greis, 2011, Quantitative evaluation of microvascular blood flow by contrast-enhanced ultrasound (CEUS), Clinical hemorheology and microcirculation 49.1-4 (2011): 137-149, incorporated by reference herein.

Analysis of the reconstructed images may be based, at least in part, on analysis of perfusion of the contrast agent relative to vasculature and/or microvasculature associated at least with the targeted tissue site. For example, the analysis may include identifying perfusion characteristics in the microvasculature, such that lesion formation is identified based on the perfusion characteristics.

In some embodiments, the console is configured to correlate perfusion characteristics within a given location of the microvasculature with physical characteristics of the microvasculature at the given location. In non-limiting examples, the characteristics analyzed may also include physical characteristics of both the contrast agent and the tissue of interest, such as flow, microflow, and stiffness. In some embodiments, the distribution and structure of the microvascular is also used as a marker of the tissue.

For example, the perfusion characteristics may include a plurality of gradations of propagation and accumulation of contrast agent into a given location of microvasculature. Accordingly, unobstructed propagation and accumulation of the contrast agent into a given location of microvasculature may be indicative of unaffected and otherwise healthy microvasculature. Similarly, lack of propagation and accumulation of contrast agent into a given location is indicative of damaged microvasculature. The damaged microvasculature may be, for example, a result of ablation. Thus, the lack of propagation and accumulation of contrast agent into the given location may be indicative of a portion of a lesion formation. Accordingly, the console may be configured to characterize a lesion formation based, at least in part, on correlation of the perfusion characteristics with physical characteristics of a given location of the microvasculature.

As disclosed herein, in non-limiting examples, the physical characteristics may be one or more of flow, microflow, and stiffness. Further, the characterization may include providing a visual indication of at least one of an extent of the lesion formation, transmurality of the lesion formation, and continuity of an ablation path associated with the lesion formation.

As disclosed herein, the console may be configured to segment a given lesion formation into at least three different regions comprising a core region, a border region immediately adjacent to and surrounding the core region, and a periphery region immediately adjacent to and surrounding the border region. The core region of a lesion formation may be associated with a complete, or near complete, lack of propagation and accumulation of contrast agent into a given location of the microvasculature and may appear normal within a 3D ultrasound image. The border region of a lesion formation may be associated with some propagation and accumulation of contrast agent into a given location of the microvasculature and presents a stronger backscatter signal within a 3D ultrasound image as compared to a backscatter signal associated with the core region. The periphery region of a lesion formation is associated with substantially unobstructed propagation and lack of accumulation of contrast agent into a given location of microvasculature and presents a weaker backscatter signal within a 3D ultrasound image as compared to backscatter signals associated with the border region a short time after injection.

The segmentation performed by the console of a given lesion formation may be based, at least in part, on a segmentation algorithm. The segmentation algorithm may include at least one of automatic thresholding, connected component analysis, and neural network.

As disclosed herein, the tissue assessment systems and methods of the invention may be used with a 4D ICE system to capture the anatomy of interest using ultrasound imaging. In specific embodiments, the invention provides systems for lesion imaging, assessment, and analysis using blood flow in the microvasculature of the myocardium to indicate and detect ablated regions. The anatomical region of interest and targeted tissue site may therefore be associated with myocardial tissue. Thus, lesion extent, before, during, and/or after an ablation procedure may be analyzed based on the ruptures/perfusion within the microvasculature.

As noted, though the systems and methods described are general and usable in treatment of problems of the vasculature, it is especially useful in the treatment of Atrial Fibrillation (AF) and other cardiac disorders as well as endovascular procedures. AF is an irregular heartbeat caused by electrical signals originating in the atrial chambers of the heart which disrupts the regular rhythm of the beating heart. AF is treated by isolating the origin of these electrical signals and limiting their transmission by ablation of the cells that surround the location of origin and conduct the electrical impulse. Systems and methods of the invention may be used for ventricular tachycardia, and general lesion monitoring such as in the denervation of renal arteries, and/or tissue assessment and analysis.

In specific embodiments, the systems use perfusion of the contrast agent to indicate and/or detect ablated regions. Exploiting the properties of ultrasound contrast agent, a contrast agent for ultrasound imaging, for example SonoVue, is injected into the coronary arteries before, during, and/or after performing one or multiple ablations.

Cardiac ablation uses energy to create lesions, i.e. scars, to block irregular electrical signals and restore a typical heartbeat. Ablations performed as part of cardiac ablation cause an alteration to the microvasculature in the myocardium, resulting in an area of the myocardium that is no longer perfused properly and thus supplied by blood, e.g., the ablation core. These differences in blood flow within the microvasculature of the myocardium are exploited for lesion detection and visualized from the reconstructed 3D image data. The reconstructed image(s) may be based, at least in part, on analysis of the level of perfusion of the contrast agent relative to vasculature and/or microvasculature associated at least with the targeted tissue site. Accordingly, the invention provides visual evidence of the extent of the lesions, in real-time during surgery, thereby allowing improved lesion path planning and execution, and improved patient outcomes.

Following propagation of the contrast agent microbubbles in the myocardium, the ablation core will not contain significant amounts of contrast agent (microbubbles) and appear normal in the ultrasound image. A second area that neighbors the ablation core, referred to as the border region, may be characterized by some thermal damage, and ruptures in the microvasculature where the contrast agent is accumulated in the tissue, thus creating a stronger backscattered signal on the ultrasound image.

The systems of the invention may capture the change before, during, and after the catheter ablation. For example, the system may include a catheter-based ultrasound imaging device operably coupled to the console and configured to transmit ultrasound pulses to, and receive echoes of the ultrasound pulses from, intravascular and/or intracardiac tissue. The ultrasound imaging device may be a four-dimensional (4D) catheter-based ultrasound imaging device. The console may be configured to receive at least full circumferential, 3D image data from the ultrasound imaging device in real, or near-real time. Further, the console may be configured to reconstruct multiple images in real, or near-real time, based, at least in part, on user input and/or predefined protocols.

In some embodiments, the console is configured to provide a 3D visualization of the anatomical region of interest and targeted tissue site during an ablation procedure being performed on the targeted tissue site.

As noted above, the system may algorithmically segment the lesion into, for example, three or more segments. The segments may be, for example, the ablation core, the border region, and the periphery. The ablation core may be delineated from the brighter-appearing border region, and the unaffected healthy region in the periphery of the lesion. Reconstructed images of these three regions, in combination with tissue oedema as an immediate response to RF ablation, may be identified using systems of the invention, for example using a 3D/4D ultrasound system in combination with the contrast agent.

For the injection of contrast agent into the blood stream, it is understood that an injection into the coronary arteries will enable a quick/efficient distribution of the contrast agent specifically into the myocardium. Alternatively, the contrast agent may be introduced also within the left atrium (enabling propagation from LA to LV to Aorta and from the aorta into the myocardium), directly in the Aorta, or other anatomical site which promote a transition of microbubbles into the myocardium. In specific embodiments, contrast agent may be injected into vasculature before and/or after performing one or more ablation procedures.

### Methods for Providing Lesion Analysis and Visualization

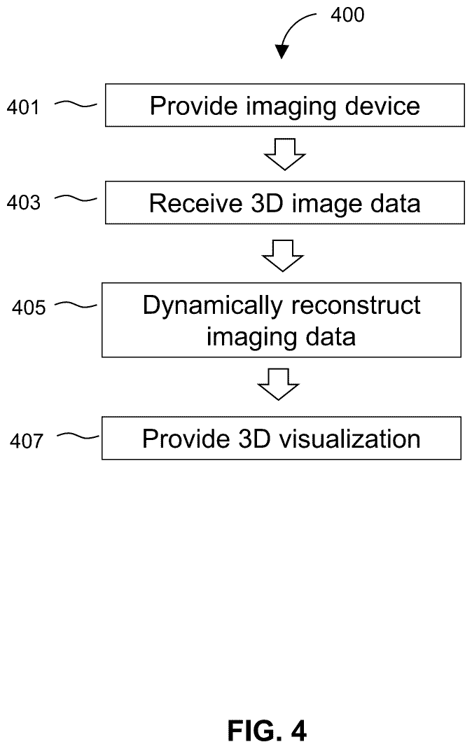

Aspects of the invention disclose methods for providing lesion analysis and visualization. FIG. 4 illustrates one embodiment of a method 400 for providing lesion analysis and visualization. The method includes providing 401 a console configured to be operably coupled to an imaging device and to communicate and exchange data with the imaging device. Further, the method includes receiving 403, via the console, three-dimensional (3D) ultrasound image data from the imaging device. The 3D image data is associated with an anatomical region of interest including a targeted tissue site and perfusion of a contrast agent therewith before, during, and/or after an ablation procedure being performed on the anatomical region of interest. The method includes dynamically reconstructing 405, via the console, multiple images from the 3D image data to provide 407 a 3D visualization of the anatomical region of interest and targeted tissue site. The 3D visualization includes visualization of any detected lesion formations in the targeted tissue site based, at least in part, on analysis of perfusion of a contrast agent relative to vasculature and/or microvasculature associated at least with the targeted tissue site.

Systems used in methods of the invention are configured to receive three-dimensional (3D) ultrasound image data from an imaging device.

In some embodiments, the invention provides for three-dimensional visualization and tissue characterization for use in minimally invasive procedures in the vasculature. For example, the tissue may be microvasculature associated with a targeted tissue site. Accordingly, ultrafast ultrasound imaging techniques, such as planewave or diverging wave imaging, may be required to enable imaging within the constraints of the application, particularly for intravascular and/or intracardiac tissue assessment and analysis. Systems and methods of the invention allow for the direct utilization of all native ultrafast imaging techniques.

For example, for intracardiac imaging, planewave imaging may refer to an ultrasound imaging modality where, through a flat transmit of all transducer elements (at different angles) from the angular imaging aperture, a plane wave front may traverse the tissue and may be partially scattered back to the transducer. From the received radio frequency (RF) (i.e. channel) data the overall image may be reconstructed at once in parallel by dynamically beamforming the received RF data for each target position.

Ultrafast ultrasound methods offer imaging at thousands of frames per second limited only by the physical propagation speed of sound waves in tissue, and enable ultrasensitive blood-flow tracking, shear-wave imaging, super-resolution imaging, and other applications. For example, achieving optimal spatial resolution while enabling artifact-free imaging of dynamic cardiac structures requires a careful balance between spatial sampling and volumetric update rate which can only be achieved using ultrafast imaging techniques. Thus, the 3D ultrasound image data received by systems of the invention may be real-time 3D ultrasound data. For example, the data may be full circumferential, three-dimensional (3D) image data. Specifically, the 3D visualization may be visualization of lesion formations in the targeted tissue site.

As disclosed herein methods of the invention may include a catheter-based ultrasound imaging device operably coupled to the console and configured to transmit ultrasound pulses to, and receive echoes of the ultrasound pulses from, intravascular and/or intracardiac tissue.

In certain embodiments of the methods, the console is configured to receive at least full circumferential, 3D image data from the ultrasound imaging device in real, or near-real time. Thus, the console is configured to reconstruct multiple images in real, or near-real time, based, at least in part, on user input and/or predefined protocols. The console is configured to provide a 3D visualization of the anatomical region of interest and targeted tissue site during an ablation procedure being performed on the targeted tissue site.

The console may be operably coupled to the imaging device and may generally control operation of the transducer probe i.e., transmission of sound waves from the probe. The console may generally include one or more processors (e.g., a central processing unit (CPU), a graphics processing unit (GPU), or both) and storage, such as main memory, static memory, or a combination of both, which communicate with each other via a bus or the like. The memory according to embodiments of the invention can include a machine-readable medium on which is stored one or more sets of instructions (e.g., software) embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

Methods of the invention may include a console in active communication with a computing system configured to communicate across a network. The computing system or computing device may include one or more processors and memory, as well as an input/output mechanism (i.e., a keyboard, knobs, scroll wheels, or the like) with which an operator can interact so as to operate the machine, including making adjustments to the transmission characteristics of the probe, saving images, and performing other tasks described herein, including selection of specific regions of interest for subsequent reconstruction into 2D and/or 3D images. During operation, the CPU and/or GPU may control the transmission and receipt of electrical currents, subsequently resulting in the emission and receipt of sound waves from the probe. The CPU and/or GPU may also analyze electrical pulses that the probe makes in response to reflected waves coming back and then may converts this data into images (i.e., ultrasound images) that can then be viewed on a display, which may be an integrated monitor. Such images may also be stored in memory and/or printed via a printer. The console may further provide control over an imaging assembly, including control over the emission of ultrasound pulses therefrom (intensity, frequency, duration, etc.) as well as control over the movement of the ultrasound transducer unit.

The computing system may include a computer program comprising an algorithm for assessing and analyzing a targeted tissue site. For example, the algorithm may be part of a computer program executable by the computing system and in communication with the console of the system. The system may be in communication with the imaging device to receive 3D ultrasound image data from the imaging device.

Imaging protocols and algorithms may be used to reconstruct properties of the targeted tissue site. The method may include one or more algorithms for dynamically reconstructing multiple images from the 3D image data to provide a 3D visualization of the anatomical region of interest and targeted tissue site. For example, using defined algorithms, systems of the method may be configured to generate the 3D visualizations based, at least in part, on analysis of perfusion of the contrast agent relative to vasculature and/or microvasculature associated at least with the targeted tissue site.

The evaluation of the vasculature/microvasculature, perfusion characteristics, and tissue characteristics using the method may include both the anatomical depiction of the vasculature/microvasculature, as well as information about the path and depth of lesions during cardiac interventions such as ablation. Specifically, data related to perfusion characteristics may be used and analyzed for visualization of an anatomical region of interest. Methods of the invention provide for analysis of perfusion characteristics within a given location of the microvasculature and identifying any lesion formation based on the perfusion characteristics. Further, the perfusion characteristics may include a plurality of gradations of propagation and accumulation of contrast agent into a given location of the microvasculature. As disclosed herein, the unobstructed propagation and accumulation of contrast agent into a given location of microvasculature may be indicative of unaffected and otherwise healthy microvasculature. Lack of propagation and accumulation of contrast agent into a given location may be indicative of damaged microvasculature as a result of ablation and thereby indicative of a portion of a lesion formation.

Characterization of lesion formation may be based, at least in part, on correlation of the perfusion characteristics with physical characteristics of a given location of the microvasculature. Thus, tissue reconstruction may be accomplished by also capturing data related to the extraction of perfusion, stiffness, strain, anisotropy, coherence, specific statistical distributions in tissue (Rayleigh, Nakagami), spectral parameters of tissue (frequency power spectrum) and other parameters. This data can be captured using a rotating three-dimensional multi-element ultrasound transducer array. Data may be captured either a single point in time, or at different stages, for example before, during, or after, an ablation procedure.

Methods of the invention may use tissue state mapping or functional imaging performed by integration of the tissue data with the appropriate imaging protocols and reconstruction algorithms, as described above. These individual protocols and algorithms may be integrated to evaluate and extract information from the data on, for example, stiffness, micro-vasculature, elasticity, perfusion, flow, shear wave speed, and other information that indicates the tissue state.

Methods of the invention dynamically reconstruct multiple images from 3D image data to provide a 3D visualization of an anatomical region of interest and targeted tissue site. The 3D visualization provides a means for assessing and analyzing an anatomical region of interest and a targeted tissue site. The assessment is based, at least in part, on analysis of perfusion of a contrast agent relative to vasculature or microvasculature associated at least with the targeted tissue site. In specific embodiments, methods of the invention provide for automated lesion segmentation using 3D/4D data acquisitions of an anatomy of interest thus facilitating detection and visualization of the full extent of lesions in the anatomy.

As disclosed herein, prior art approaches to lesion imaging attempt to characterize changes in ultrasound data that are related to tissue property changes, such as stiffness and echogenicity, with little success. The invention discloses a novel method for imaging scarring and/or necrosis of tissue by exploiting characteristic changes in the microvasculature. Specifically, the methods use tissue perfusion imaging to indicate and/or detect regions of scar and/or necrosis.

As disclosed, the 3D ultrasound image data may be real-time 3D ultrasound data, and the 3D visualization may include visualization of lesion formations in the targeted tissue site. Thus, the tissue assessment systems and methods of the invention may use a 4D ICE system that captures the anatomy of interest using ultrasound imaging. Specifically an ultrasound contrast agent may be used to visualize a targeted tissue site through a change in perfusion to the tissue.

Ultrasound contrast agent is comprised of small echogenic bubbles in the size of red blood cells, as noted in Greis, 2011, Quantitative evaluation of microvascular blood flow by contrast-enhanced ultrasound (CEUS); Clinical hemorheology and microcirculation 49.1-4:137-149, incorporated in its entirety herein. Generally, ultrasound contrast agents consist of tiny gas-filled microbubbles the size of red blood cells. As is known to persons skilled in the art, due to their size distribution, they are purely intravascular tracers which may be used for imaging blood distribution and flow. Thus, the contrast agent can effectively propagate into both larger vessels and the microvasculature. More generally, the contrast agent can propagate into any vessel where red blood cells can be present.

Ultrasound contrast agents alter the reflection pattern, for example by dramatically increasing backscatter signal, and resonating in a linear manner when acoustic pressure is applied. With increasing acoustic pressure, nonlinear vibrational patterns appear. Specifically, the microbubbles oscillate when being compressed by the effect of positive pressure created by the ultrasound waves and they expand in the negative pressure phase. The compression of the gas is greater than expansion which creates a non-linear response (echo). This greatly affects ultrasound backscatter and increases vascular contrast.

The current invention exploits these properties to provide novel methods for using tissue perfusion, e.g. blood flow in an anatomical region of interest, to detect regions of scar or necrosis in the tissue. Specifically, an ultrasound contrast agent is used to visualize tissue properties through a change in perfusion to tissue. Differences in perfusion within the microvasculature are exploited for novel tissue analysis and visualization.

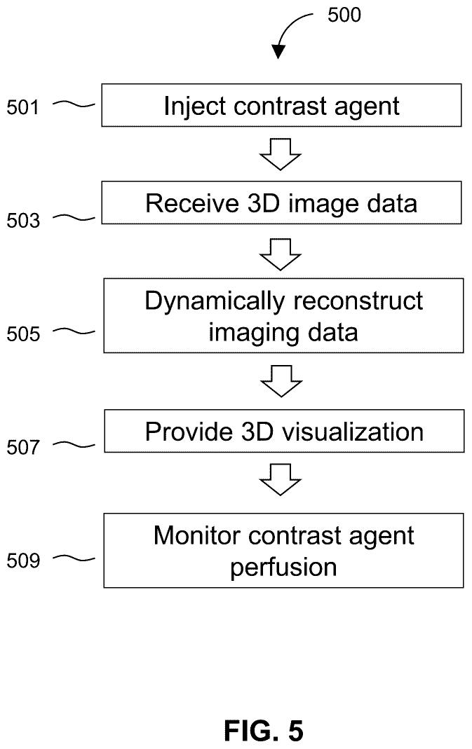

FIG. 5 is block diagram of a method 500 for contrast-enhanced imaging according to one embodiment of the invention. The method 500 includes injecting 501 the contrast agent, receiving 503 3D image data, dynamically reconstructing 505 the image data, providing 507 a 3D visualization of an anatomical region of interest, and monitoring 509 the perfusion of the contrast agent. As discussed in more detail herein, the contrast agent may be injected into the heart directly, or indirectly though an artery or vein. Injection of the contrast agent may occur one or more times, for example as it relates to imaging an anatomical region of interest and/or to suit the needs of the procedure performed. The contrast agent is monitored via ultrasound imaging. Monitoring may include monitoring the flush of the contrast agent, the perfusion of the contrast agent, and/or the dispersion of the contrast agent. Monitoring via ultrasound imaging may be performed at any time, for example at injection of the contrast agent, before and/or during a procedure, and post-follow-up after a procedure. Monitoring may include displaying changes in perfusion of the contrast agent. Reconstructing may include reconstructing multiple images from the 3D data to provide a 3D visualization of the anatomical region of interest and targeted tissue site. Accordingly, reconstruction may include algorithmically relating changes in the contrast agent perfusion to tissue properties.

In some embodiments, the method includes analysis of the reconstructed images. For example, analysis of the reconstructed images may be based, at least in part, on analysis of perfusion of the contrast agent relative to vasculature or microvasculature associated at least with the targeted tissue site. For example, the analysis may include identifying perfusion characteristics in the microvasculature, such that lesion formation is identified based on the perfusion characteristics.

The lesions may be visualized in real time. For example, the contrast agent may be injected when the catheter is positioned to view the anatomy of interest. Thus, the anatomy of interest may be viewed while the contrast agent is injected. Accordingly, it is possible to create a 3D anatomical map that shows the tissue before and after a procedure, such as an ablation procedure, to allow for a comparative view. In this application, the ultrasound data may be gated using electrocardiogram (ECG) signals and 3D maps computed before and after the ablation of the anatomy of interest.

As disclosed herein, the invention provides a system for receiving three-dimensional (3D) ultrasound image data from an imaging device. The console may be configured to receive at least full circumferential, 3D image data from the ultrasound imaging device in real, or near-real time. Further, the console may be configured to reconstruct multiple images in real, or near-real time, based, at least in part, on user input and/or predefined protocols.

The system provides for dynamically reconstructing multiple images from the 3D image data to provide a 3D visualization of the anatomical region of interest and targeted tissue site.

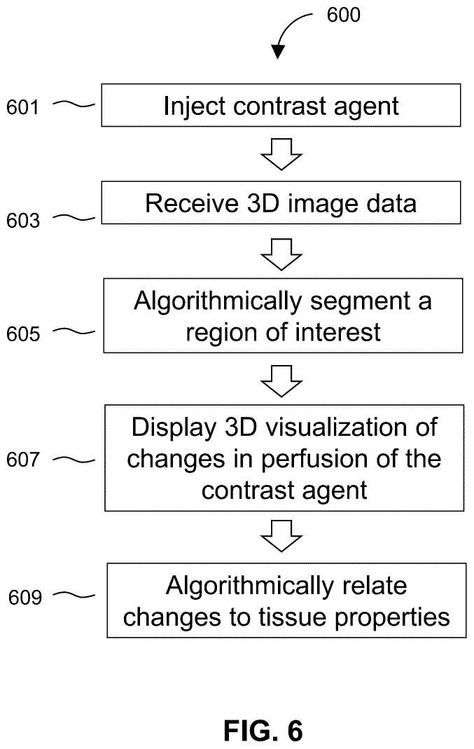

FIG. 6 is a block diagram of a method 600 for contrast-enhanced imaging according to one embodiment of the invention. As described in more detail below, the method may generally include injecting 601 the contrast agent, receiving 603 3D image data, segmenting 605 a region of interest, displaying 607 a visualization of changes in perfusion of the contrast agent, and relating 609 the changes in perfusion of the contrast agent to tissue properties.

As described herein, the 3D image data may generally include full circumferential, 3D image data, specifically 3D volumetric data, captured by the imaging device during an ultrasound procedure. The 3D image data may be associated with an anatomical region of interest including a targeted tissue site and perfusion of a contrast agent within the targeted tissue site. Thus, in some embodiments, the 3D visualization include visualization of lesion formations in the targeted tissue. For example, the tissue may be microvasculature associated with the targeted tissue site.

In specific embodiments, the methods may algorithmically segment the anatomical region of interest, such as a lesion, into, for example, three or more segments. The segments may include, for example, the core, the border region, and the periphery. The core may be delineated from the brighter-appearing border region, and the unaffected healthy region in the periphery of the lesion. Reconstructed images of these three regions are identified using systems of the invention, for example using a 3D/4D ultrasound system in combination with the contrast agent. The segmentation may be performed by a vision algorithm such as thresholding, connected component analysis or neural network based segmentation. The contrast agent in the intact vasculature disappears quickly, but the particles that exit through ruptured vasculature remain long enough for imaging.

The console may be configured to provide a 3D visualization of the anatomical region of interest and targeted tissue site during an ablation procedure being performed on the targeted tissue site. It is important to note that, while specific examples of systems of the invention are directed toward analysis and assessment of ablated regions of the myocardium, the targeted tissue may be any tissue wherein perfusion characteristics of a contrast agent may be evaluated. As described in detail herein, the distribution and/or lack of microvasculature perfusion may be assessed as a marker for tissue in general, including but not limited to, lesion imaging.

Specifically, following propagation of the contrast agent microbubbles in the tissue, an area of scarring and/or necrosis, i.e. a lesion, referred to as the core, will not contain significant amounts of contrast agent (microbubbles) and appear normal in the ultrasound image. A second area, that neighbors the core, referred to as the border region, may be characterized by some thermal damage, and ruptures in the microvasculature where the contrast agent is accumulated in the tissue, thus creating a stronger backscattered signal on the ultrasound image. Systems of the invention capture the changes in perfusion of the contrast agent and algorithmically relate these changes to the tissue properties. Accordingly, in some embodiments, the analysis includes identifying perfusion characteristics in the microvasculature.

In specific embodiments, the methods may algorithmically segment the lesion into, for example, three or more segments. The segments may include, for example, the core, the border region, and the periphery. The core may be delineated from the brighter-appearing border region, and the unaffected healthy region in the periphery of the lesion. Reconstructed images of these three regions are identified using systems of the invention, for example using a 3D/4D ultrasound system in combination with the contrast agent. The segmentation may be performed by a vision algorithm such as thresholding, connected component analysis or neural network based segmentation. The contrast agent in the intact vasculature disappears quickly, but the particles that exit through ruptured vasculature remain long enough for imaging.

The lesions may be visualized in real time. For example, the contrast agent may be injected when the catheter is positioned to view the anatomy of interest. Thus, the anatomy of interest may be viewed while the contrast agent is injected. Accordingly, it is possible to create a 3D anatomical map that shows the tissue before and after an ablation procedure. This allows for a comparative view. In this application, the ultrasound data may be gated using electrocardiogram (ECG) signals and 3D maps computed before and after the ablation of the anatomy of interest

For injection of contrast agent into the blood stream, an injection of the contrast agent directly within the heart or coronary arteries may be made. An injection directly into the coronary arteries enables a quick and efficient distribution of the contrast agent specifically into the myocardium. Alternatively, the contrast agent may be introduced within the left atrium. This enables propagation of the contrast agent from the left atrium (LA) (enabling propagation from the LA to left ventricle (LV) to Aorta and from the aorta into the myocardium), directly in the Aorta, or other anatomical site which promote a transition of microbubbles into the myocardium, as described in Greis, 2011, Quantitative evaluation of microvascular blood flow by contrast-enhanced ultrasound (CEUS), Clinical hemorheology and microcirculation 49.1-4 (2011): 137-149, incorporated by reference herein.

Analysis of the reconstructed images is based, at least in part, on analysis of perfusion of the contrast agent relative to vasculature or microvasculature associated at least with the targeted tissue site. For example, the analysis may include identifying perfusion characteristics in the microvasculature, such that lesion formation is identified based on the perfusion characteristics.

In some embodiments, the console is configured to correlate perfusion characteristics within a given location of the microvasculature with physical characteristics of the microvasculature at the given location. In non-limiting examples, the characteristics analyzed may also include physical characteristics of both the contrast agent and the tissue of interest, such as flow, microflow, and stiffness. In some embodiments, the distribution and structure of the microvascular is also used as a marker of the tissue.

For example, the perfusion characteristics may include a plurality of gradations of propagation and accumulation of contrast agent into a given location of microvasculature. Accordingly, unobstructed propagation and accumulation of the contrast agent into a given location of microvasculature is indicative of unaffected and otherwise healthy microvasculature. Similarly, lack of propagation and accumulation of contrast agent into a given location is indicative of damaged microvasculature. The damaged microvasculature may be, for example, a result of ablation. Thus, the lack of propagation and accumulation of contrast agent into the given location may be indicative of a portion of a lesion formation. Accordingly, the console may be configured to characterize a lesion formation based, at least in part, on correlation of the perfusion characteristics with physical characteristics of a given location of the microvasculature. As disclosed herein, in non-limiting examples, the physical characteristics may be one or more of flow, microflow, and stiffness. Further, the characterization may include providing a visual indication of at least one of an extent of the lesion formation, transmurality of the lesion formation, and continuity of an ablation path associated with the lesion formation.

The console may be configured to segment a given lesion formation into at least three different regions comprising a core region, a border region immediately adjacent to and surrounding the core region, and a periphery region immediately adjacent to and surrounding the border region. The core region of a lesion formation may be associated with a complete, or near complete, lack of propagation and accumulation of contrast agent into a given location of the microvasculature and may appear normal within a 3D ultrasound image. The border region of a lesion formation may be associated with some propagation and accumulation of contrast agent into a given location of the microvasculature and presents a stronger backscatter signal within a 3D ultrasound image as compared to a backscatter signal associated with the core region. The periphery region of a lesion formation is associated with substantially unobstructed propagation and lack of accumulation of contrast agent into a given location of microvasculature and presents a weaker backscatter signal within a 3D ultrasound image as compared to backscatter signals associated with the border region a short time after injection.

The segmentation performed by the console of a given lesion formation may be based, at least in part, on a segmentation algorithm. The segmentation algorithm may include at least one of automatic thresholding, connected component analysis, and neural network.

As disclosed herein, the tissue assessment methods of the invention may be used with a 4D ICE system to capture the anatomy of interest using ultrasound imaging. In specific embodiments, the invention provides systems for lesion imaging, assessment, and analysis using blood flow in the microvasculature of the myocardium to indicate and detect ablated regions. The anatomical region of interest and targeted tissue site may therefore be associated with myocardial tissue. Thus, lesion extent, before, during, and/or after an ablation procedure may be analyzed based on the ruptures/perfusion within the microvasculature.

As noted, though the methods described herein are general and usable in treatment of problems of the vasculature, it is especially useful in the treatment of Atrial Fibrillation (AF) and other cardiac disorders as well as endovascular procedures. AF is an irregular heartbeat caused by electrical signals originating in the atrial chambers of the heart which disrupts the regular rhythm of the beating heart. AF is treated by isolating the origin of these electrical signals and limiting their transmission by ablation of the cells that surround the location of origin and conduct the electrical impulse. Systems and methods of the invention may be used for ventricular tachycardia, and general lesion monitoring such as in the denervation of renal arteries, and/or tissue assessment and analysis.

In specific embodiments, the methods use perfusion of the contrast agent to indicate and/or detect ablated regions. Exploiting the properties of ultrasound contrast agent, a contrast agent for ultrasound imaging, for example SonoVue, is injected into the coronary arteries before, during, and/or after performing one or multiple ablations. Cardiac ablation uses energy to create lesions, i.e. scars, to block irregular electrical signals and restore a typical heartbeat. Ablations performed as part of cardiac ablation cause an alteration to the microvasculature in the myocardium, resulting in an area of the myocardium that is no longer perfused properly and thus supplied by blood, e.g., the ablation core. These differences in blood flow within the microvasculature of the myocardium are exploited for lesion detection and visualized from the reconstructed 3D image data. The reconstructed image(s) may be based, at least in part, on analysis of the level of perfusion of the contrast agent relative to vasculature and/or microvasculature associated at least with the targeted tissue site. Accordingly, the invention provides visual evidence of the extent of the lesions, in real-time during surgery, thereby allowing improved lesion path planning and execution, and improved patient outcomes.

Following propagation of the contrast agent microbubbles in the myocardium, the ablation core will not contain significant amounts of contrast agent (microbubbles) and appear normal in the ultrasound image. A second area, that neighbors the ablation core, referred to as the border region, may be characterized by some thermal damage, and ruptures in the microvasculature where the contrast agent is accumulated in the tissue, thus creating a stronger backscattered signal on the ultrasound image.

The methods of the invention may capture the change before, during, and after the catheter ablation. For example, the system may include a catheter-based ultrasound imaging device operably coupled to the console and configured to transmit ultrasound pulses to, and receive echoes of the ultrasound pulses from, intravascular and/or intracardiac tissue. The ultrasound imaging device may be a four-dimensional (4D) catheter-based ultrasound imaging device. The console may be configured to receive at least full circumferential, 3D image data from the ultrasound imaging device in real, or near-real time. Further, the console may be configured to reconstruct multiple images in real, or near-real time, based, at least in part, on user input and/or predefined protocols.

In some embodiments, the console is configured to provide a 3D visualization of the anatomical region of interest and targeted tissue site during an ablation procedure being performed on the targeted tissue site.

As noted above, the system may algorithmically segment the lesion into, for example, three or more segments. The segments may be, for example, the ablation core, the border region, and the periphery. The ablation core may be delineated from the brighter-appearing border region, and the unaffected healthy region in the periphery of the lesion. Reconstructed images of these three regions, in combination with tissue oedema as an immediate response to RF ablation, are identified using systems of the invention, for example using a 3D/4D ultrasound system in combination with the contrast agent.

For the injection of contrast agent into the blood stream, it is understood that an injection into the coronary arteries will enable a quick/efficient distribution of the contrast agent specifically into the myocardium. Alternatively, the contrast agent may be introduced also within the left atrium (enabling propagation from LA to LV to Aorta and from the aorta into the myocardium), directly in the Aorta, or other anatomical site which promote a transition of microbubbles into the myocardium. In specific embodiments, contrast agent may be injected into vasculature before and/or after performing one or more ablation procedures.

As used in any embodiment herein, the term “module” may refer to software, firmware and/or circuitry configured to perform any of the aforementioned operations. Software may be embodied as a software package, code, instructions, instruction sets and/or data recorded on non-transitory computer readable storage medium. Firmware may be embodied as code, instructions, or instruction sets and/or data that are hard-coded (e.g., nonvolatile) in memory devices. “Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. The modules may, collectively or individually, be embodied as circuitry that forms part of a larger system, for example, an integrated circuit (IC), system on-chip (SoC), desktop computers, laptop computers, tablet computers, servers, smartphones, etc.

Any of the operations described herein may be implemented in a system that includes one or more storage mediums having stored thereon, individually or in combination, instructions that when executed by one or more processors perform the methods. Here, the processor may include, for example, a server CPU, a mobile device CPU, and/or other programmable circuitry.

Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, crasable programmable read-only memories (EPROMs), electrically erasable programmable read-only memories (EEPROMs), flash memories, Solid State Disks (SSDs), magnetic or optical cards, or any type of media suitable for storing electronic instructions. Other embodiments may be implemented as software modules executed by a programmable control device. The storage medium may be non-transitory.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A system for providing tissue analysis and visualization, the system comprising:
a console comprising a hardware processor coupled to non-transitory, computer-readable memory containing instructions executable by the processor to cause the console to:
receive three-dimensional (3D) ultrasound image data from an imaging device, the 3D image data being associated with an anatomical region of interest including a targeted tissue site and perfusion of a contrast agent therewith before, during, and/or after an ablation procedure being performed thereon; and
dynamically reconstruct multiple images from the 3D image data to provide a 3D visualization of the anatomical region of interest and targeted tissue site, based, at least in part, on analysis of perfusion of the contrast agent relative to vasculature associated at least with the targeted tissue site.

2. The system of claim 1, wherein the 3D ultrasound image data is real-time 3D ultrasound data.

3. The system of claim 1, wherein the 3D visualization comprises visualization of lesion formations in the targeted tissue site.

4. The system of claim 1, wherein the tissue comprises microvasculature associated with the targeted tissue site.

5. The system of claim 4, wherein the analysis comprises identifying perfusion characteristics in the microvasculature.

6. The system of claim 5, wherein a lesion formation is identified based on the perfusion characteristics.

7. The system of claim 5, wherein the console is configured to correlate perfusion characteristics within a given location of the microvasculature with physical characteristics of the microvasculature at said given location.

8. The system of claim 5, wherein the perfusion characteristics comprise a plurality of gradations of propagation and accumulation of contrast agent into a given location of microvasculature.

9. The system of claim 8, wherein:
unobstructed propagation and accumulation of contrast agent into a given location of microvasculature is indicative of unaffected and otherwise healthy microvasculature; and
lack of propagation and accumulation of contrast agent into a given location is indicative of damaged microvasculature.

10. The system of claim 9, wherein the damaged microvasculature is a result of ablation and the lack of propagation and accumulation of contrast agent into the given location is indicative of a portion of a lesion formation.

11. The system of claim 10, wherein the console is configured to characterize a lesion formation based, at least in part, on correlation of the perfusion characteristics with physical characteristics of a given location of the microvasculature.

12. The system of claim 11, wherein the physical characteristics are one or more of flow, microflow, and stiffness.

13. The system of claim 11, wherein characterization comprises providing a visual indication of at least one of an extent of the lesion formation, transmurality of the lesion formation, and continuity of an ablation path associated with the lesion formation.

14. The system of claim 11, wherein the console is configured to segment a given lesion formation into at least three different regions comprising a core region, a border region immediately adjacent to and surrounding the core region, and a periphery region immediately adjacent to and surrounding the border region.

15. The system of claim 14, wherein a core region of a lesion formation is associated with a complete, or near complete, lack of propagation and accumulation of contrast agent into a given location of the microvasculature and appears normal within a 3D ultrasound image.

16. The system of claim 14, wherein a border region of a lesion formation is associated with some propagation and accumulation of contrast agent into a given location of the microvasculature and presents a stronger backscatter signal within a 3D ultrasound image as compared to a backscatter signal associated with the core region.

17. The system of claim 14, wherein a periphery region of a lesion formation is associated with substantially unobstructed propagation and lack of accumulation of contrast agent into a given location of microvasculature and presents a weaker backscatter signal within a 3D ultrasound image as compared to backscatter signals associated with the border region a short time after injection.

18. The system of claim 17, wherein the console performs segmentation of a given lesion formation based, at least in part, on a segmentation algorithm.

19. The system of claim 18, wherein the segmentation algorithm comprises at least one of automatic thresholding, connected component analysis, and neural network-based segmentation.

20. The system of claim 1, wherein the contrast agent is injected into vasculature before and/or after performing one or more ablation procedures.

21. The system of claim 1, further comprising a catheter-based ultrasound imaging device operably coupled to the console and configured to transmit ultrasound pulses to, and receive echoes of the ultrasound pulses from, intravascular and/or intracardiac tissue.

22. The system of claim 21, wherein the ultrasound imaging device comprises a four-dimensional (4D) catheter-based ultrasound imaging device.

23. The system of claim 21, wherein the console is configured to receive at least full circumferential, 3D image data from the ultrasound imaging device in real, or near-real time, and the console is configured to reconstruct multiple images in real, or near-real time, based, at least in part, on user input and/or predefined protocols.

24. The system of claim 23, wherein the console is configured to provide a 3D visualization of the anatomical region of interest and targeted tissue site during an ablation procedure being performed on the targeted tissue site.

25. The system of claim 1, wherein the anatomical region of interest and targeted tissue site are associated with myocardial tissue.
